(19) United States
(12) Patent Application Publication
(10) Pub. No.: US 2005/0217599 A1
(43) Pub. Date: Oct. 6, 2005

(54) Saddlebox pet carrier product

(76) Inventor: Michael Varner, Chicago, IL (US)

Correspondence Address: DAVID J. BREMER, 4921 N. DAMEN AVENUE, CHICAGO, IL, 60625, US

(21) Appl. No.: 10/813,777

(22) Filed: Mar. 31, 2004

(51) Int. Cl. C12Q 1/68

(52) U.S. Cl. 119/496

(57) ABSTRACT

The invention is an animal carrier product for carrying an animal on a cycle. The product is mounted to the cycle at a saddlebag position to provide a low center of gravity while the cycle is in use. The product is mounted at the saddlebag position to enable the cycle to carry a human passenger in a typical passenger position behind a cycle driver. Some embodiments of the product are configured to mount interchangeably with a commercially-available saddlebag cargo container, utilizing the mounting bracket provided with the commercially-available saddlebag cargo container.

The invention is an animal carrier product for carrying an animal on a cycle. The product is mounted to the cycle at a saddlebag position. Mounting the product at the saddlebag position results in a low center of gravity that is beneficial to the stability of the cycle when the cycle is in use. Furthermore, mounting the product at the saddlebag position enables the cycle to carry a human passenger in a typical passenger position behind a cycle driver.

The product has an exposed section and a sheltered section for providing sheltered and exposed locations, respectively, for the animal. The product is designed so that the animal can move autonomously between the sections without intervention from the outside.

Since the animal is free to move autonomously between the exposed section and the sheltered section, the cycle driver is not required to monitor the position of the animal to ensure the animal is comfortable and content while the product is in use.

In some embodiments, the product is configured to attach to a bracket that is pre-attached to the cycle, for example a custom-designed saddlebox-mounting bracket.

Alternatively, in some embodiments the product is configured to attach to a pre-attached commercially-available mounting bracket, for example, the bracket supplied with a commercially available saddlebag cargo carrier for mounting the saddlebag cargo container to a cycle.

In some embodiments, the product is designed to mount interchangeably with the saddlebag cargo container. The interchangeability of the product with the saddlebag cargo container eliminates the need for an additional animal carrier product mounting bracket.

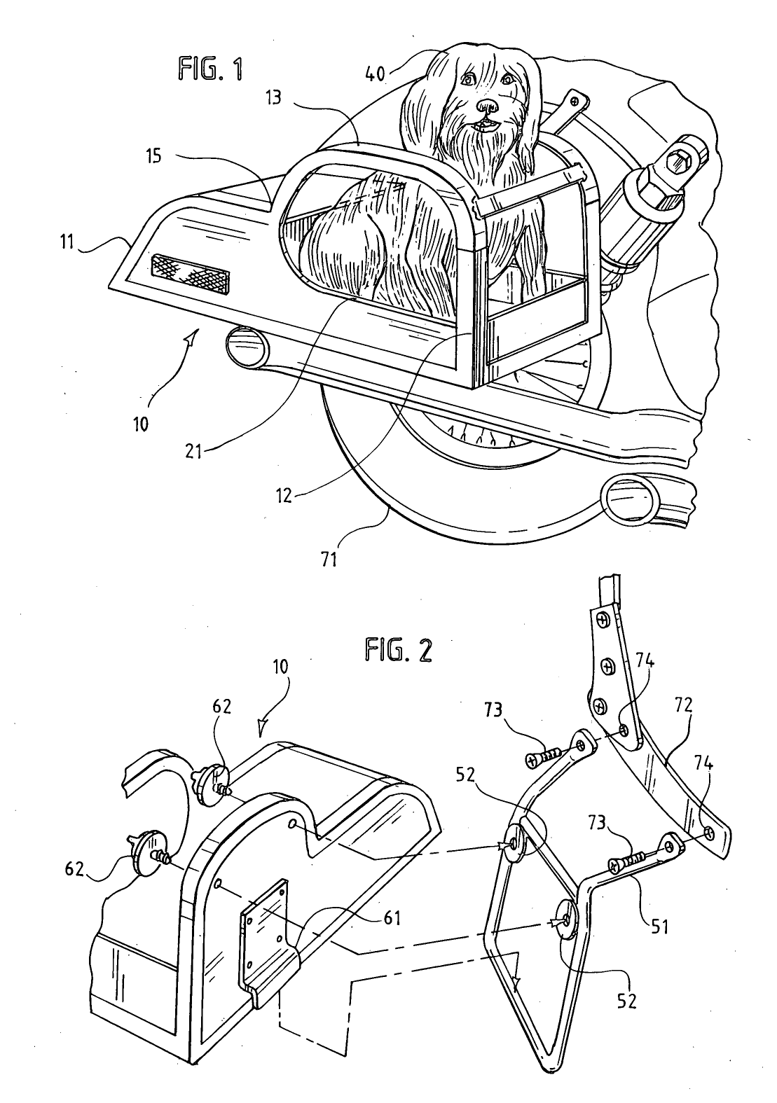

FIG. 1 depicts a perspective view of the product in use mounted to a cycle.

FIG. 2 depicts an exploded perspective view of the product configured to mount to a pre-attached, commercially available, saddlebag bracket.

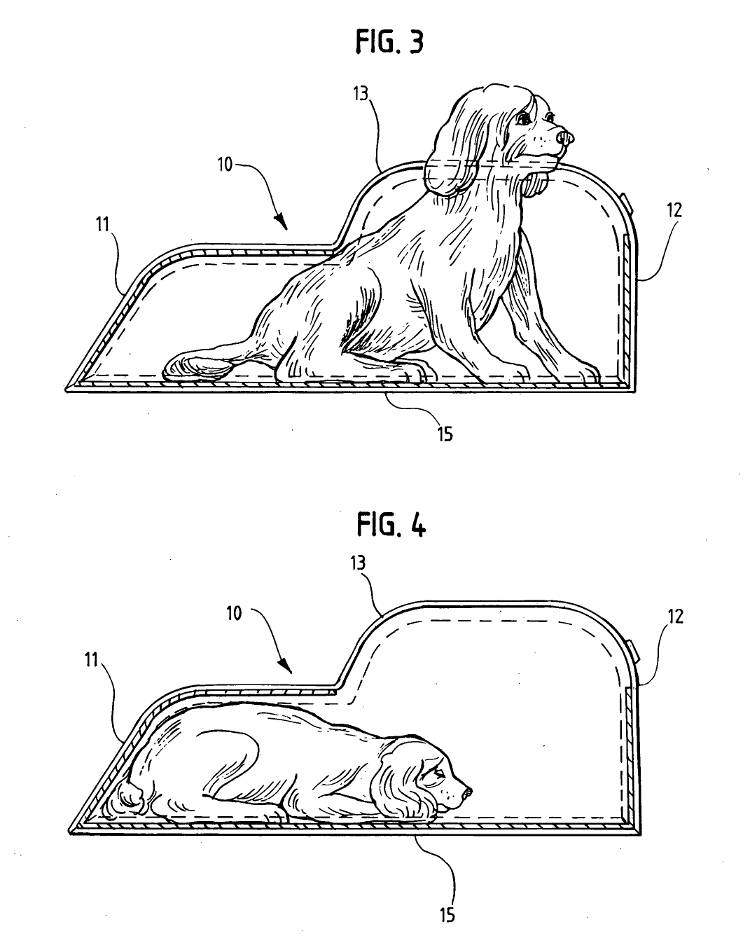

FIG. 3 depicts a side section view of the product showing the animal in the exposed section of the product.

FIG. 4 depicts a side section view of the product showing the animal in the sheltered section of the product.

The saddlebag position is derived from the bags that would be hung across hindquarters of a horse behind the saddle. Early saddlebags were leather and canvas cargo bags. The saddlebags could be connected together by a strap that extended across the horse hindquarters so that the saddlebags would hang down on either side of the horse.

The saddlebag application was later adapted so that the saddlebags could be mounted to wheeled cycles. Similar to the horse-mounted saddlebags, early wheeled cycle saddlebags could extend across the top of the cycle near a wheel and hang down on either side of the wheel. The early cycle saddlebags could also utilize an additional mounting element that could attach between the bag and the cycle and prevent the bag from becoming entangled in the wheel while in use.

The saddlebag application further evolved so that the saddlebags were no longer connected by a strap stretched over the top of the cycle. Modem saddlebags are mounted to the cycle structure so that the saddlebag is positioned alongside of the wheel and substantially below the top of the cycle and proximal the wheel.

The saddlebag position, as used here and throughout to describe the mounting position of the animal carrier product, refers to the mounting position typified by modern, commercially-available saddlebag cargo containers. It is recognized that the saddlebag position may evolve further in unforeseen ways and still maintain the aforementioned functionality.

As shown in FIG. 1, the animal carrier product 10 comprises a saddlebox 15 for holding the animal 40. The saddlebox 15 in use contains the animal and can inhibit accidental and unwanted animal escapes.

The saddlebox can be rigid, flexible, resilient and combinations thereof. The saddlebox can be made from various materials as long as the materials are sufficiently strong to contain the animal when the saddlebox is in use.

The saddlebox can be made from plastics and other polymeric materials. Alternatively, the saddlebox can be made from metal, for example steel and aluminum. Alternatively, the saddlebox can be made from composite materials such as fiberglass and resins. Alternatively, the saddlebox can be made from flexible materials such as leather, canvas, woven nylon, and various other flexible materials. Alternatively, the saddlebox can be made from combinations of the aforementioned materials and various other materials that have sufficient strength to contain the animal.

The saddlebox can have various shapes and sizes. As shown in FIG. 1, the saddlebox 15 can have substantially flat sides. Alternatively, the saddlebox can be egg-shaped, ellipsoid, arbitrarily-shaped, and various other shapes and combinations thereof, so long as the saddlebox can contain the animal.

The saddlebox 15 comprises an exposed section 12 where the animal is at least partially exposed to the outside environment.

In FIG. 3, the product 10 is shown with the animal in the exposed section 12. In this embodiment, the exposed section has an open top 13.

Other embodiments can expose the animal in various ways so long as the saddlebox confines the animal and can inhibit accidental and unwanted animal escapes.

For example, the exposed section can have a closed top and open side areas. Alternatively, the exposed section can have porous areas, for example screened, grated, and barred areas, so that the animal is exposed to the outside environment and contained in the saddlebox while the product is in use.

The saddlebox also comprises a sheltered section 11 where the animal is sheltered from the outside environment.

The sheltered section provides the animal with some protection from wind, noise, precipitation, and other aspects of the outside environment.

Especially when the cycle is in use, some animals may prefer the more protected environment of the sheltered section to the exposed section.

In FIG. 4, the product 10 is shown with the animal in the sheltered section 11. In this embodiment the sheltered section has a closed top and substantially closed sides.

The exposed section 12 and the sheltered section 11 must connect in a way that enables the animal to autonomously move from the sheltered section to the exposed section and back again while the product is in use.

For example, if the animal is uncomfortable in the exposed section, the animal can autonomously move into the sheltered section, without requiring outside intervention to facilitate movement from one section to the other. Similarly, the animal can autonomously move into the exposed section from the sheltered section.

By enabling the animal to move autonomously, the product can minimize driver distraction while the product is in use, since the driver is not required to monitor the actions of the animal to ensure the animal appears comfortable and content in the saddlebox.

In the embodiment shown in FIG. 1, the exposed section 12 and the sheltered section 11 transition smoothly into each other. The exposed section and the sheltered section can connect in various ways, so long as the animal can autonomously move between the sections without requiring outside intervention to facilitate moving between sections.

For example, the sections can connect via a channel, a swinging door, a revolving door, a connection with an automatically controlled opening, and various other types of connections.

The product is designed to mount on a cycle at the aforementioned saddlebag position.

In FIG. 1, the product 10 is mounted at the saddlebag position proximal a rear wheel 71. Other embodiments can mount at the saddlebag position proximal a front wheel.

In FIG. 1, the product is shown with the exposed section towards a front of the cycle. Other embodiments can have the sheltered sections towards the front of the cycle.

The product comprises mounting means for mounting the product to a cycle at the saddlebag position.

The mounting means can be integral to the product. For example, the product can have saddlebox connectors formed into the saddlebox that are configured to correspond with cycle connectors that are attached to cycle structure.

Alternatively, the cycle connectors can be integral to the cycle.

Alternatively, the saddlebox connectors can be separate from the saddlebox and designed to mount the saddlebox by interfacing with the cycle connectors.

Alternatively, the mounting means can comprise a custom-made bracket that interfaces with the cycle structure and positions the product at the saddlebag position.

Alternatively, the mounting means can be configured to connect to a bracket that is pre-attached to the cycle. For example, the product can be configured to mount interchangeably with a commercially-available saddlebag cargo container, utilizing the commercially-available saddlebag bracket 51 provided for the saddlebag cargo container.

The mounting means can correspond to connections provided by a commercially-available saddlebag bracket 51 for mounting the commercially-available saddlebag cargo container.

In the embodiment shown in FIG. 2, the product 10 mounts to a pre-attached commercially-available saddlebag bracket 51. The saddlebag bracket 51 is attached to the cycle structure 72 by threaded fasteners 73 that correspond to threaded passages 74 on the cycle structure. Other commercially-available saddlebag brackets can utilize various means for attaching to the cycle.

The saddlebag bracket 51 shown in FIG. 2 is comparable to a Cruiseliner Saddlebag Mounts Part No. KIT-SB103, commercially-available from National Cycle, Inc. Similar brackets are designed to mount to a structure of a specific make and model motorcycle. Similar brackets provide an interface between connections provided on the cycle structure and standardized connections provided on certain commercially-available saddlebag cargo carriers.

In one embodiment of the product, the product mounting means can be configured to correspond to the standardized connections provided on certain commercially-available saddlebag cargo carriers. This embodiment of the product can mount to the saddlebag bracket interchangeably with the saddlebag cargo containers.

In FIG. 2, the mounting means comprise a hook 61 for hooking to the pre-attached saddlebag bracket 51.

In FIG. 2, the mounting means further comprise a saddlebox fastener 62 for connecting to a bracket fastener 52. The bracket fastener 52 is a standardized connector provided as part of the commercially-available saddlebag bracket 51. The bracket fastener 52 is configured to correspond to the standardized connection provided on the commercially-available saddlebag cargo container.

The saddlebox can have a liner that covers interiorly at least a part of the saddlebox. The liner can provide additional and different interior properties to at least the part of the saddlebox.

For example, the liner can be resilient, waterproof, absorbent, cushioned, non-skid, and various other forms and combinations thereof.

The liner can be a coating applied interiorly to the saddlebox and the liner can be separate from and placed inside the saddlebox.

As shown in FIG. 1, the saddlebox 15 can comprise a window 21. The window 21 is connected to the saddlebox and can be integral to the saddlebox.

The window can be part of the exposed section and the window can be part of the sheltered section.

The window is substantially transparent. The window can enable the animal to view the outside environment while the product is in use.

For some animals, viewing the outside environment, while contained in the saddlebox, can be calming. For some animals, viewing the outside environment, while contained in the saddlebox, can minimize motion sickness symptoms.

The product can comprise a deflector for deflecting wind and sunlight from entering the saddlebox.

The deflector can be connected to the saddlebox. Alternatively, the deflector can be connected to the cycle. Alternatively, the deflector can be connected to the mounting means, bracket, and various other locations.

The product can comprise restraint means for restraining while the product is in use.

The restraint means can further inhibit unwanted and accidental animal escapes, especially from the exposed section.

The restraint means can comprise a restraint connector attached to the saddlebox. The restraint connector can connect to an animal leash and an animal harness to restrain an animal from escaping from the exposed section.

The restraint means can comprise screen, netting, mesh and other porous materials covering the open top and other open areas of the saddlebox.

1. An animal carrier product for carrying an animal on a cycle, the product being mounted to the cycle at a saddlebag position for increased stability and to accommodate a human passenger while the product is in use, the product comprising:
a saddlebox for holding the animal, the saddlebox comprising:
an exposed section,
the exposed section for exposing the animal to the outside environment;
a sheltered section,
the sheltered section for sheltering the animal from the outside environment;
the exposed section and the sheltered section being connected together so that the animal can move autonomously from the exposed section to the sheltered section and so that the animal can move autonomously from the sheltered section to the exposed section, while the product is in use; and
mounting means for mounting the product to the cycle at the saddlebag position.

2. The animal carrier product of claim 1 wherein the saddlebox further comprises:
a window,
the window being connected to the exposed section;
the window being substantially transparent;
the window enabling the animal to view the outside environment.

3. The animal carrier product of claim 1 wherein the product further comprises:
a deflector,
the deflector being connected to the product so that the deflector deflects direct sunlight and deflects strong wind from entering the exposed section.

4. An animal carrier product for carrying an animal on a cycle, the product being mounted to the cycle at a saddlebag position for increased stability and to accommodate a human passenger while the product is in use, the product comprising:
a saddlebox for holding the animal, the saddlebox comprising:
an exposed section,
the exposed section for exposing the animal to the outside environment;
a sheltered section,
the sheltered section for sheltering the animal from the outside environment;
the exposed section and the sheltered section being connected together so that the animal can move autonomously from the exposed section to the sheltered section and so that the animal can move autonomously from the sheltered section to the exposed section, while the product is in use; and mounting means for mounting the product to the cycle at the saddlebag position,
the mounting means being configured to enable the saddlebox to mount interchangeably with a commercially-available saddlebag cargo container, the commercially-available saddlebag cargo container having standardized connections.

5. The animal carrier product of claim 4 wherein the saddlebox further comprises:
a window,
the window being connected to the exposed section;
the window being substantially transparent;
the window enabling the animal to view the outside environment.

6. The animal carrier product of claim 4 wherein the product further comprises:
a deflector,
the deflector being connected to the product so that the deflector deflects direct sunlight and deflects strong wind from entering the exposed section.

7. (canceled)

8. (canceled)

9. (canceled)
